(19) United States
(12) Patent Application Publication
(10) Pub. No.: US 2017/0259006 A1
(43) Pub. Date: Sep. 14, 2017

(54) MECHANISM FOR SETTING A MAXIMUM DOSE OF A MEDICAMENT FOR A VARIABLE DOSE DRUG DELIVERY DEVICE

(71) Applicant: Sanofi, Paris (FR)

(72) Inventors: Richard James Vincent Avery, Warwick, Warwickshire (GB); Matthew Meredith Jones, Warwick, Warwickshire (GB); William Geoffrey Arthur Marsh, Warwick, Warwickshire (GB); Anthony Paul Morris, Warwick, Warwickshire (GB); David Aubrey Plumptre, Warwick, Warwickshire (GB); Samuel Keir Steel, Warwick, Warwickshire (GB); James Alexander Senior, Warwick, Warwickshire (GB); Robert Frederick Veasey, Warwick, Warwickshire (GB)

(73) Assignee: Sanoi, Paris (FR)

(21) Appl. No.: 15/528,623

(22) PCT Filed: Nov. 24, 2015
(86) PCT No.: PCT/EP2015/077512

§ 371 Date: May 22, 2017

(30) Foreign Application Priority Data
Nov. 24, 2014 (EP) 14306861.7

(51) Int. Cl. A61M 5/315; A61M 5/32; A61M 5/24

(52) U.S. Cl. CPC A61M 5/31538; A61M 5/24; A61M 5/329; A61M 5/31553; A61M 5/31585; A61M 5/3157; A61M 2005/3154; A61M 2005/3126

(57) ABSTRACT

The present disclosure concerns a mechanism for setting a dose of a medicament for a variable dose drug delivery device, comprising: a housing, a sleeve component configured to rotate relative to the housing during a dose setting operation, and a stop member which is configured to limit a total amount of possible rotation of the sleeve component to a maximum and, thereby, to limit a maximum settable dose, wherein the stop member is configured to allow varying the maximum which determines the total amount of possible rotation of the sleeve component.

### CROSS-REFERENCE TO RELATED APPLICATIONS

This application is a U.S. national stage application under 35 USC §371 of International Application No. PCT/EP2015/077512, filed on Nov. 24, 2015, which claims priority to European Patent Application No. 14306861.7 filed on Nov. 24, 2014, the entire contents of which are incorporated herein by reference.

### TECHNICAL FIELD

The present disclosure is generally directed to a mechanism for setting a dose of a medicament for a variable dose drug delivery device.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament.

### SUMMARY

The present disclosure is not directed to so called fixed dose devices, which only allow dispensing of a predefined dose without the possibility to increase or decrease the set dose.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. The present disclosure is applicable for both types of devices, i.e. for disposable devices as well as for reusable devices.

These types of pen delivery devices (so named because they often resemble an enlarged fountain pen) generally comprise three primary elements: a cartridge section that includes a cartridge often contained within a housing or holder; a needle assembly connected to one end of the cartridge section; and a dosing section connected to the other end of the cartridge section. A cartridge (often referred to as an ampoule) typically includes a reservoir that is filled with a medication (e.g., insulin), a movable rubber type bung or stopper located at one end of the cartridge reservoir, and a top having a pierceable rubber seal located at the other, often necked-down, end. A crimped annular metal band is typically used to hold the rubber seal in place. While the cartridge housing may be typically made of plastic, cartridge reservoirs have historically been made of glass.

The needle assembly is typically a replaceable double-ended needle assembly. Before an injection, a replaceable double-ended needle assembly is attached to one end of the cartridge assembly, a dose is set, and then the set dose is administered. Such removable needle assemblies may be threaded onto, or pushed (i.e., snapped) onto the pierceable seal end of the cartridge assembly.

The dosing section or dose setting mechanism is typically the portion of the pen device that is used to set (select) a dose. During an injection, a spindle or piston rod contained within the dose setting mechanism presses against the bung or stopper of the cartridge. This force causes the medication contained within the cartridge to be injected through an attached needle assembly. After an injection, as generally recommended by most drug delivery device and/or needle assembly manufacturers and suppliers, the needle assembly is removed and discarded.

A further differentiation of drug delivery device types refers to the drive mechanism: There are devices which are manually driven, e.g. by a user applying a force to an injection button, devices which are driven by a spring or the like and devices which combine these two concepts, i.e. spring assisted devices which still require a user to exert an injection force. The spring-type devices involve springs which are preloaded and springs which are loaded by the user during dose selecting. Some stored-energy devices use a combination of spring preload and additional energy provided by the user, for example during dose setting.

However, common drug delivery devices in general have a fixed maximum settable dose. For different kinds of medicaments, e.g. insulin, GLP-1 or heparin, the common doses differ significantly from each other. Moreover, even for the same medicament the requirements for different patients may require different maximum settable doses for each patient. Accordingly, there is a need for drug delivery devices varying in their respective maximum settable dose.

The present disclosure provides an improved mechanism for setting a dose of a medicament for a variable drug delivery device. In particular, the mechanism may be configured for allowing to vary a maximum settable dose of a drug delivery device.

According to the disclosure, a mechanism for setting a dose of a medicament for a variable drug delivery device is proposed which comprises a housing, a sleeve component configured to rotate relative to the housing during a dose setting operation and a stop member which is configured to limit a total amount of possible rotation of the sleeve component to a maximum and, thereby, to limit a maximum settable dose, wherein the stop member is configured to allow varying the total amount of possible rotation of the sleeve component. Accordingly, the stop member is configured to allow varying the maximum settable dose.

Accordingly, the mechanism is configured such that a maximum settable dose can be varied. Thus, a drug delivery device comprising the mechanism can be used to deliver a wide range of medicaments, including those in which a maximum dose has been defined while minimizing manufacturing complexity. The same components can be assembled using substantially similar production equipment, but varying the maximum settable dose via the stop member. The present disclosure allows the use of the same kind of drug delivery device for different kinds of medicaments and, further, for patients with different dose requirements.

For example, a first drug delivery device may have a first maximum settable dose and a second drug delivery device may have a second maximum settable dose which is different from the first maximum settable dose. The first and the second drug delivery device may only differ in the setting of their respective stop member. In particular, the stop member of the first drug delivery device may allow a total amount of possible rotation of the sleeve component relative to the housing different from a total amount of possible rotation of the sleeve component relative to the housing in the second drug delivery device.

The variable dose drug delivery device may be a drug delivery device wherein a dose is user-settable. The user can set a to-be-delivered dose to one of multiple possible numbers.

The housing may be a housing of the drug delivery device. Accordingly, the housing may be an outer component of the drug delivery device. In particular, the sleeve component and the stop member may be arranged at least partially inside the housing.

The sleeve component may be a number sleeve. In particular, numbers or other dosing indicia may be provided on an outer surface of the sleeve component. For example, the sleeve component may be configured such that one of said numbers is visible through a window of the housing wherein said visible number corresponds to a currently set dose. However, in alternate embodiments, the sleeve component may be free of numbers and other dosing indicia. The sleeve component may be configured to rotate relative to the housing in a first rotational direction during the dose setting operation and, further, to rotate relative to the housing in a second rotational direction opposite to the first rotational direction during a dose dispense operation.

A user may operate the sleeve component either directly or by operating a further component, e.g. a dose selector, which transfers a user input into a motion of the sleeve component.

The sleeve component may be configured such that each rotation of the sleeve component in the first rotational direction relative to the housing corresponds to an increase in the currently set dose. By limiting the total amount of possible rotation of the sleeve component, the stop member effectively limits the maximum settable dose. In particular, the stop member may be configured to limit the total amount of possible rotation of the sleeve component relative to the housing in the first rotational direction. The total amount of possible rotation may be a whole number or a fraction, so that the stop member may be configured to limit the total angle of rotation.

A low total amount of possible rotation corresponds to a low maximum settable dose. Vice versa, a high total amount of possible rotation corresponds to a high maximum settable dose.

The stop member may be an element separate from the sleeve component and the housing. The stop member may be engageable and disengageable from the sleeve component. Alternatively or additionally, the stop member may be engageable and disengageable from the housing.

The stop member is configured to allow varying the total amount of possible rotation. However, the mechanism may be configured such that the total amount of possible rotation may not be varied during a dose setting operation or during a dose dispense operation. Instead, a separate operation has to be carried out to vary the total amount of possible rotation. In particular, the mechanism may be configured such that a user is prevented from varying the total amount of possible rotation. Further, the mechanism may be configured such that only the manufacturer or a health care professional may be allowed to vary the total amount of possible rotation of the sleeve component.

Further, the mechanism may also be suitable for dispensing the set dose. A dose setting operation may be defined as an operation wherein the drug delivery device is transferred into a state of being ready to dispense a defined dose, i.e. a defined sub-quantity of a medicament in a container of the drug delivery device. The dose dispense operation may further be defined as the operation wherein the previously set dose is dispensed.

According to one embodiment, the sleeve component comprises a thread, wherein the mechanism further comprises a nut member that is engaged with the thread such that it is configured to travel along the thread during the dose setting operation, and wherein the stop member is configured to limit a rotation of the sleeve component by limiting a movement of the nut member relative to the sleeve component.

In particular, the nut member may be a gauge element. The nut member may be configured to cooperate with the sleeve component or with the housing to display the currently set dose.

The thread may be configured such that the nut member can only carry out a concurrently rotational and axial movement relative to the sleeve component. The thread may be a helical thread. The nut member may be configured to travel along the thread in a first axial direction and in a first rotational direction relative to the sleeve component during a dose setting operation. Further, the nut member may be configured to travel along the thread in a second axial direction opposite to the first axial direction and in a second rotational direction opposite to the first rotational direction relative to the sleeve component during a dose dispense operation.

The stop member may be configured to limit a movement of the nut member relative to the sleeve component by blocking either an axial movement of the nut member in the first axial direction relative to the sleeve component or by blocking a rotational movement of the nut member in the first rotational direction relative to the sleeve component after a rotation corresponding to the total amount of possible rotation of the sleeve component relative to the housing has been carried out.

In one embodiment, the stop member may be configured to directly engage with the nut member. In particular, the sleeve component, the nut member and the stop member may cooperate with each other such that a further rotation of the sleeve component in a first rotational direction is prevented when the stop member engages with the nut member. The stop member may be configured to directly engage with the nut member by abutting the nut member and, thereby, preventing a further movement of the nut member in a direction beyond the point of abutment.

In one embodiment, the stop member may be engaged with the thread of the sleeve component, the total amount of possible rotation of the sleeve component relative to the housing may be determined by the position of the stop member along the thread, and the total amount of possible rotation can be varied by moving the stop member along the thread. In particular, when the stop member moves further distally along the thread, the total amount of possible rotation may be reduced.

In particular, the stop member may be engaged with the sleeve component such that the stop member may not be moved accidentally relative to the sleeve component during the operation of the drug delivery device. Further, the stop member may be engaged with the sleeve component such that it is possible to disengage the stop member from the sleeve component and/or to move the stop member relative to the sleeve component.

The stop member and the thread may each comprise a ratchet feature allowing a rotation of the stop member along the thread during manufacture of the mechanism and preventing a rotation of the stop member in an assembled drug delivery device.

In particular, the stop member may be an internal member of the assembled drug delivery device such that it is not accessible to a user of the drug delivery device. Thereby, a user may be prevented from changing the position of the stop member along the thread. This embodiment allows a manufacturer to determine the maximum settable dose. Thus, it is easy for the manufacturer to manufacture a multitude of drug delivery devices, each having a different maximum settable dose by varying the position of the stop member along the thread. Drug delivery devices comprising the same components can therefore be used for multiple medicaments or drug regimens with minimal implications or alterations required during the design of parts, logistics, or assembly line.

In one embodiment, the stop member and the thread may each comprise ratchet features allowing a rotation of the stop member along the thread in a first rotational direction and preventing a rotation of the stop member in a second rotational direction opposite to the first rotational direction. Thus, these ratchet features may prevent that the maximum settable dose is overridden during an operation of the device when an excessive force is applied to the stop member or the sleeve component.

In one embodiment, the stop member may comprise an abutment surface and the stop member may be configured to limit an axial movement of the nut member in the first axial direction relative to the sleeve component when the nut member abuts the abutment surface of the stop member. The first axial direction may be a proximal direction. The first axial direction may be a dose incrementing direction or a dose setting direction.

The abutment surface of the stop member may be a distally facing surface.

In particular, the nut member may be constrained such that it can only carry out a concurrently rotational and axial movement relative to the sleeve component along the thread. When the axial part of the concurrently axial and rotational movement is blocked, it is not possible to increase the set dose any further.

The abutment surface of the stop member may be arranged such that it is configured to be abutted by an end face of the nut member facing in a circumferential direction or to be abutted by a protrusion of the nut member protruding in a radially outward direction.

The stop member may be configured to engage with the housing in two different orientations wherein each orientation corresponds to a different total amount of possible rotation. Thus, the stop member may define different maximum settable doses depending on its orientation when engaged with the housing. This allows to vary the total amount of possible rotation of the sleeve member relative to the housing by engaging the stop member in a different orientation with the housing. Accordingly, a simple way to vary the maximum settable dose is provided.

The stop member may comprise a first engagement feature and the housing may comprise a second engagement feature, wherein the first engagement feature of the stop member is directly engageable with the second engagement feature of the housing and wherein the first and the second engagement features are configured to allow an axial movement of the stop member relative to the housing when the first and the second engagement features are engaged. In particular, the first and the second engagement feature may be configured to allow an axial movement of the stop member in a second axial direction relative to the housing and to prevent an axial movement of the stop member in the first direction relative to the housing. The second axial direction may be a distal direction. The second axial direction may be a dose decrementing direction or a dose dispensing direction.

Accordingly, the engagement features may be configured to allow decreasing of the maximum settable dose but prevent increasing of the maximum settable dose. Alternatively, the engagement features may prevent increasing of the maximum settable dose when the stop member is locked, but allow a different maximum settable dose to be selected when the stop member is unlocked. Further, the first and the second engagement features may be configured to prevent a rotational movement of the stop member relative to the housing when the first and the second engagement features are engaged.

The mechanism may further comprise at least a second stop member comprising a second abutment surface wherein the housing is configured to be engaged with exactly one of the stop members, wherein each of the stop members defines a different total amount of possible rotation of the sleeve component relative to the housing depending on the axial position of the abutment surface of the respective stop member. Thus, each stop member defines a different maximum settable dose. The maximum settable dose can be varied by removing one stop member from the housing and by engaging the housing with another stop member.

The stop member may be configured such that the total amount of possible rotation can only be varied by engaging the stop member with a specific tool. Thereby a user is prevented from accidentally changing the maximum number of settable doses.

The nut member may be configured to rotate in a first rotational direction relative to the sleeve component during the dose setting operation and the nut member may further be configured to rotate in a second rotational direction opposite to the first rotational direction relative to the sleeve component during a dose dispensing operation. Accordingly, at the end of the dose dispensing operation, the nut member may be in its initial position relative to the sleeve component. At any given moment, the relative position of the nut member and the sleeve component may correspond to a currently set dose.

The mechanism may further comprise a minimum dose stop feature defining a minimum dose which is configured such that a dose dispensing operation can only be carried if a set dose is at least as big as the minimum dose.

The stop member and the second stop member may each comprise a transparent material. In particular, the stop member may be a lens component which is configured to be arranged over a window in the housing.

The stop member may not be visible from the outside of the drug delivery device. The stop member may instead be an internal member.

The stop member may be retained inside the housing. Alternatively, the stop member may be configured to be assembled to the remainder of the mechanism from the outside of the housing.

According to a second aspect, the present disclosure concerns a drug delivery device comprising the above-described mechanism. The drug delivery device may by a variable dose drug delivery device. Moreover, the drug delivery device may comprise the functional and structural features disclosed herein with respect to the mechanism.

The term “medicament”, as used herein, preferably means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivatives are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as

Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra-low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two p sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while p and E have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystallizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### BRIEF DESCRIPTION OF THE FIGURES

Non-limiting, exemplary embodiments of the disclosure will now be described with reference to the accompanying drawings, in which:

FIG. 1 shows a top view of the drug delivery device of the present disclosure in the minimum dose position;

FIG. 2 shows an exploded view of the components of the device of FIG. 1;

FIG. 3 shows a sectional view of the device of FIG. 1;

FIG. 4a shows an enlarged sectional view of a detail of the device of FIG. 1 in the dose setting mode;

FIG. 4b shows an enlarged sectional view of a detail of the device of FIG. 1 in the dose dispensing mode;

FIG. 5 shows an interface between the sleeve component and the button of the device of FIG. 1;

FIG. 6 shows an interface between the housing and the button of the device of FIG. 1;

FIGS. 7a and 7b show an interface between the sleeve component and the drive sleeve of the device of FIG. 1 in the dose setting mode and in the dose dispensing mode;

FIG. 8 shows an interface between the piston rod and a bearing of the device of FIG. 1;

FIG. 9 shows an interface between the clutch plate and the button of the device of FIG. 1;

FIG. 10 shows in a sectional view the components of an end of dose clicker of the device of FIG. 1;

FIGS. 11a-c show in enlarged views the sequence of generating a click at the end of dose dispensing of the device of FIG. 1;

FIGS. 12a-c show in enlarged sectional views the sequence of generating a click at the end of dose dispensing of the device of FIG. 1;

FIG. 13 shows the nut member of the device of FIG. 1;

FIG. 14 shows a portion of the sleeve component of the device of FIG. 1;

FIG. 15 shows a further portion of the sleeve component of the device of FIG. 1;

FIG. 16 shows a portion of the drive spring of the device of FIG. 1;

FIGS. 17a and 17b show top views of the device of FIG. 1 with 0 units dialed and with 96 units dialed;

FIG. 18 shows an interface between the housing and the drive sleeve of the device of FIG. 1;

FIG. 19 shows an interface between the clutch plate and the drive sleeve of the device of FIG. 1;

FIG. 20 shows a last dose mechanism of the device of FIG. 1;

FIG. 21 shows the torsion spring of the device of FIG. 1; and

FIGS. 22a-c show different embodiments of the threads between the piston rod and the housing of the device of FIG. 1.

FIG. 23 shows an enlarged view of a part of a sleeve component according a first preferred embodiment.

FIG. 24 shows a perspective view of a stop member according to the first preferred embodiment.

FIG. 25 shows the stop member being assembled to the sleeve component.

FIG. 26 shows the nut member interacting with the stop member thereby limiting the maximum settable dose.

FIG. 27 shows a perspective view of the housing according to a second preferred embodiment.

FIG. 28 shows the stop member according to the second preferred embodiment.

FIG. 29 and FIG. 30 each show a cross-sectional view of the assembly comprising the sleeve component, the stop member and the nut member.

FIG. 31 shows a perspective view of the housing according to a third preferred embodiment.

FIG. 32 shows the stop member according to a third preferred embodiment.

FIGS. 33 and 34 each show cross-sectional views of the assembly comprising the housing, the nut member, the sleeve component and a stop member.

### DETAILED DESCRIPTION

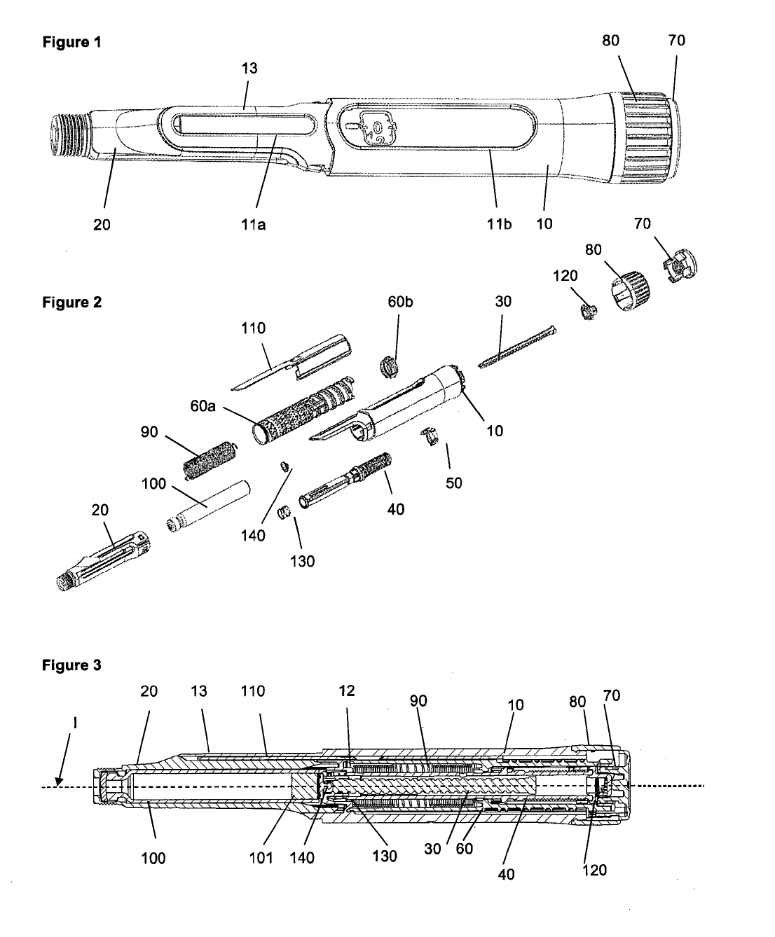

FIG. 1 shows a drug delivery device in the form of an injection pen. The device has a distal end (left end in FIG. 1) and a proximal end (right end in FIG. 1). The component parts of the drug delivery device are shown in FIG. 2. The drug delivery device comprises a body or housing 10, a cartridge holder 20, a piston rod 30, a drive sleeve 40, a nut 50, a sleeve component 60, a button 70, a dial grip or dose selector 80, a torsion spring 90, a cartridge 100, a gauge element or nut member 110, a clutch plate 120, a clutch spring 130 and a bearing 140. A needle arrangement (not shown) with a needle hub and a needle cover may be provided as additional components, which can be exchanged as explained above. All components are located concentrically about a common principal axis I of the mechanism which is shown in FIG. 3.

The piston rod 30 may be a lead screw. The nut member 110 may be a gauge element.

The sleeve component 60 may be a number sleeve. In particular, the sleeve component 60 may be a dose indicator. Numbers, e.g. dose numbers corresponding to a currently set dose, or other dosing indicia may be provided on the sleeve component 60.

The housing 10 or body is a generally tubular element having a proximal end with an enlarged diameter. The housing 10 provides location for the liquid medication cartridge 100 and cartridge holder 20, windows 11a, 11b for viewing the dose number on the sleeve component 60 and the nut member 110 or gauge element, and a feature on its external surface, e.g. a circumferential groove, to axially retain the dose selector 80. A flange-like or cylindrical inner wall 12 comprises an inner thread engaging the piston rod 30. The housing 10 further has at least one internal, axially orientated slot or the like for axially guiding the nut member 110. In the embodiment shown in the Figures, the distal end is provided with an axially extending strip 13 partly overlapping cartridge holder 20. The Figures depict the housing 10 as a single housing component. However, the housing 10 could comprise two or more housing components which may be permanently attached to each other during assembly of the device.

The cartridge holder 20 is located at the distal side of housing 10 and permanently attached thereto. The cartridge holder may be a transparent or translucent component which is tubular to receive cartridge 100. The distal end of cartridge holder 20 may be provided with means for attaching a needle arrangement. A removable cap (not shown) may be provided to fit over the cartridge holder 20 and may be retained via clip features on the housing 10.

The piston rod 30 is rotationally constrained to the drive sleeve 40 via a splined interface. When rotated, the piston rod 30 is forced to move axially relative to the drive sleeve 40, through its threaded interface with the inner wall 12 of housing 10. The piston rod 30 is an elongate member with an outer thread 31 (FIG. 3) engaging the corresponding thread of the inner wall 12 of housing 10. The thread 31 may have a large lead-in, for example a wedge shape form, at its distal end to engage a corresponding housing thread form on the first rotation. The interface comprises at least one longitudinal groove or track and a corresponding protrusion or spline 45 of the drive sleeve 40. At its distal end, the piston rod 30 is provided with an interface for clip attachment of the bearing 140. In the present embodiment, this interface comprises two clip arms 32 extending in the distal direction defining an insertion space between them for insertion of a bearing 140 interface. As an alternative, the interface may comprise only one single clip arm extending more than 180° about the longitudinal axis, or may comprise one or several clip arms 32. The clip arm(s) 32 may have a bent form with a recessed clip portion as shown in FIG. 8. Preferably, the clip arm(s) form a cylindrical outer face having a diameter equal to or smaller than the outer diameter of the piston rod 30 at the base of the groove (flute base) of the outer thread 31. A concave contact surface 33 is provided between the clip arms 32 for abutment of a corresponding portion of bearing 140.

The drive sleeve 40 is a hollow member surrounding the piston rod 30 and arranged within sleeve component 60. It extends from an interface with the clutch plate 120 to the contact with the clutch spring 130. The drive sleeve 40 is axially movable relative to the housing 10, the piston rod 30 and the sleeve component 60 in the distal direction against the bias of clutch spring 130 and in the opposite proximal direction under the bias of clutch spring 130.

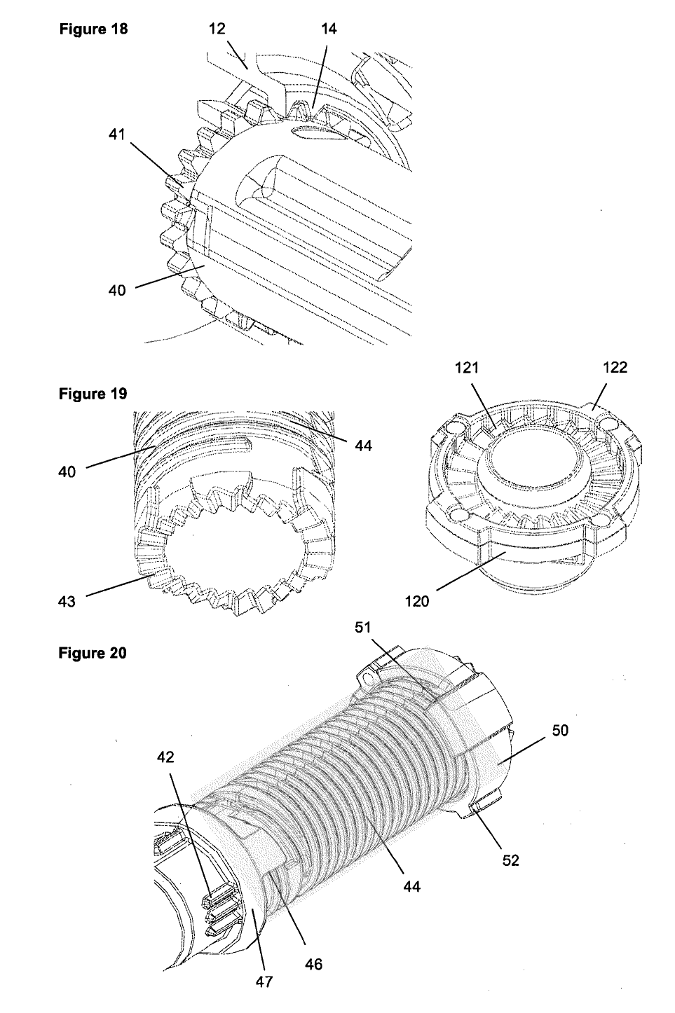

A splined tooth interface with the housing 10 prevents rotation of the drive sleeve 40 during dose setting. This interface which is shown in FIG. 18 in detail comprises a ring of radially extending outer teeth 41 at the distal end of drive sleeve 40 and corresponding radially extending inner teeth 14 of the housing component 10. When the button 70 is pressed, these drive sleeve 40 to housing 10 spline teeth 14, 41 are disengaged allowing the drive sleeve 40 to rotate relative to housing 10.

A further splined tooth interface with the sleeve component 60 is not engaged during dialing, but engages when the button 70 is pressed, preventing relative rotation between the drive sleeve 40 and sleeve component 60 during dispense. In the preferred embodiment shown in FIGS. 7a and 7b this interface comprises inwardly directed splines 61 on a flange 62 on the inner surface of the sleeve component 60 and a ring of radially extending outer splines 42 of drive sleeve 40. The corresponding splines 61, 42 are located on the sleeve component 60 and the drive sleeve 40, respectively, such that axial movement of the drive sleeve 40 relative to the (axially fixed) sleeve component 60 engages or disengages the splines to rotationally couple or decouple the drive sleeve 40 and the sleeve component 60.

Preferably, the splines 61, 42 are arranged such that they are decoupled when teeth 41 of drive sleeve 40 and inner teeth 14 of housing component 10 mesh and engage when teeth 41 and inner teeth 14 disengage. In a preferred embodiment the splines 61, 42 are longer in the axial direction compared with teeth 41, 14. This allows engagement of the splines 61, 42 shortly before disengagement of teeth 41, 14. In other words, the splines 61, 42 and the teeth 41, 14 are designed and arranged such that actuation of the button 70 rotationally constrains the drive sleeve 40 to the sleeve component 60 before the drive sleeve 40 is allowed to rotate relative to housing 10. Similarly, as the button 70 is released after dose dispensing axial movement of the drive sleeve 40 first rotationally constrains the drive sleeve 40 to the housing and thereafter decouples splines 61, 42. As an alternative to the corresponding splines 61, 42 teeth may be provided. As a further alternative or in addition to splines 61, 42, drive sleeve 40 and sleeve component 60 may be rotationally coupled to each other during dose dispensing via clutch plate 120.

An interface of the drive sleeve 40 which is shown in FIG. 19 comprises a ring of ratchet teeth 43 located at the proximal end face of drive sleeve 40 and a ring of corresponding ratchet teeth 121 of clutch plate 120.

The drive sleeve 40 has a threaded section 44 providing a helical track for the nut 50 (FIG. 20). In addition, a last dose abutment or stop 46 is provided which may be the end of the thread 44 track or preferably a rotational hard stop for interaction with a corresponding last dose stop 51 of nut 50, thus limiting movement of the nut 50 on the thread 44. At least one longitudinal spline 45 engages a corresponding track of the piston rod 30. Further, the drive sleeve 40 is provided with a ramp 47 interacting with a clicker arm 67 when the drive sleeve 40 is in its distal position during dose dispensing, i.e. when button 70 is depressed.

The last dose nut 50 is located between the sleeve component 60 and the drive sleeve 40. It is rotationally constrained to the sleeve component 60, via a splined interface (splines 52 on nut 50). It moves along a helical path relative to the drive sleeve 40, via a threaded interface (thread 44), when relative rotation occurs between the sleeve component 60 and drive sleeve 40 which is during dialing only. This is shown in FIG. 20. As an alternative, the nut 50 may be splined to the drive sleeve 40 and threaded to the sleeve component 60. In the embodiment shown in the Figures, the nut 50 is a full nut, but in alternative embodiments it may be a half nut, i.e. a component extending approximately 180° around the center axis of the device. A last dose stop 51 is provided engaging stop 46 of drive sleeve 40 when a dose is set corresponding to the remaining dispensable amount of medicament in the cartridge 100.

The sleeve component 60 is a tubular element as shown in FIGS. 2 and 3. The sleeve component 60 is rotated during dose setting (via dose selector 80) and dose correction and during dose dispensing by torsion spring 90. Together with nut member 110 the sleeve component 60 defines a zero position (‘at rest’) and a maximum dose position. Thus, the sleeve component 60 may be seen as a dose setting member.

For manufacturing reasons, the sleeve component 60 of the embodiment shown in the Figures comprises a sleeve component lower 60a which is rigidly fixed to a sleeve component upper 60b during assembly to form the sleeve component 60. Sleeve component lower 60a and sleeve component upper 60b are separate components only to simplify sleeve component 60 mold tooling and assembly. As an alternative, the sleeve component 60 may be a unitary component. The sleeve component 60 is constrained to the housing 10 by features towards the distal end to allow rotation but not translation. The sleeve component lower 60a is marked with a sequence of numbers, which are visible through the nut member 110 and the openings 11a, 11b in the housing 10, to denote the dialed dose of medicament.

Further, the sleeve component lower 60a has a portion with an outer thread 63 engaging the nut member 110. End stops 64, 65 are provided at the opposite ends of thread 63 to limit relative movement with respect to the nut member 110.

Clutch features which have the form of a ring of splines 66 in the embodiment of FIG. 5 are provided inwardly directed on sleeve component upper 60b for engagement with splines 73 of the button 70 during dose setting and dose correction. A clicker arm 67 is provided on the outer surface of sleeve component 60 which interacts with the drive sleeve 40 and the nut member 110 for generating a feedback signal. In addition, the sleeve component lower 60a is rotationally constrained to the nut 50 and to the clutch plate 120 via a splined interface comprising at least one longitudinal spline.

An interface for attachment of the torsion spring 90 to the sleeve component lower 60a comprises large lead-ins and a groove feature 68 with a pocket 69 or anchor point for receiving a first coil or hook portion of the spring. The groove 68 has an end feature in the form of a ramp that is in interference with the hook portion 91 of the spring. The design of the groove 68 is such that the spring 90 may be received within the pocket 69 without interfering with the nut member 110.

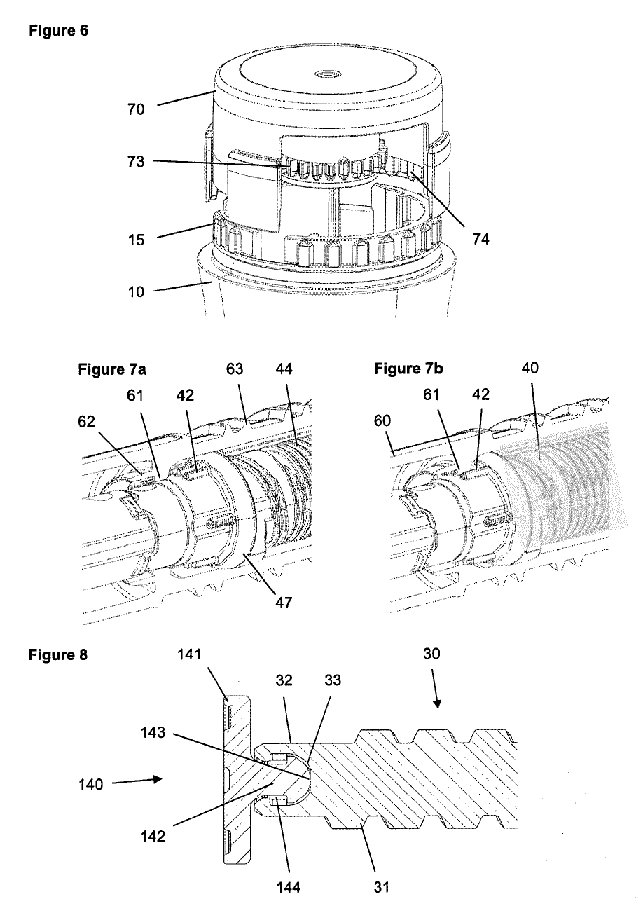

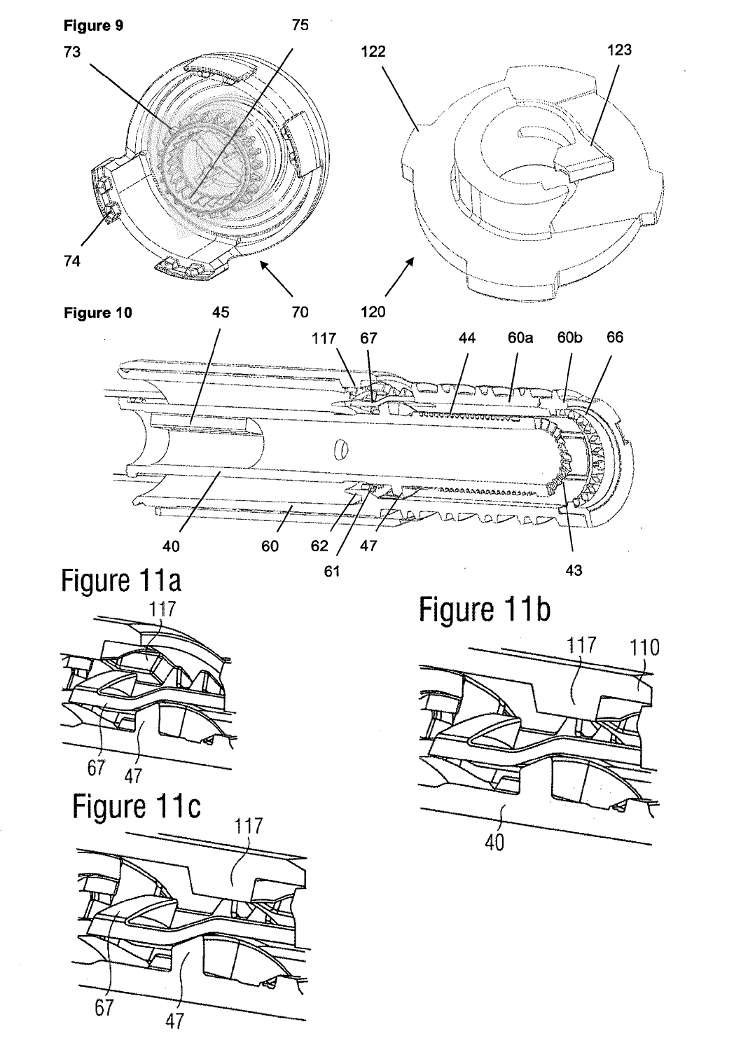

The button 70 which forms the proximal end of the device is permanently splined to the dose selector 80. A central stem 71 extends distally from the proximal actuation face of the button 70. The stem 71 is provided with a flange 72 carrying the splines 73 for engagement with splines 66 of the sleeve component upper 60b (FIG. 5). Thus, it is also splined via splines 66, 73 (FIG. 5) to the sleeve component upper 60b when the button 70 is not pressed, but this spline interface is disconnected when the button 70 is pressed. The button 70 has a discontinuous annular skirt with splines 74. When the button 70 is pressed, splines 74 on the button 70 engage with splines on the housing 10 (FIG. 6), preventing rotation of the button 70 (and hence the dose selector 80) during dispense. These splines 74, 15 disengage when the button 70 is released, allowing a dose to be dialed. Further, a ring of ratchet teeth 75 is provided on the inner side of flange 72 (FIG. 9) for interaction with clutch plate 120.

The dose selector 80 is axially constrained to the housing 10. It is rotationally constrained, via the splined interface, to the button 70. This splined interface which includes grooves interacting with spline features formed by the annular skirt of button 70 remains engaged irrespective of the dose button 70 axial positions. The dose selector 80 or dose dial grip is a sleeve-like component with a serrated outer skirt.

The torsion spring 90 is attached at its distal end to the housing 10 and at the other end to the sleeve component 60. The torsion spring 90 is located inside the sleeve component 60 and surrounds a distal portion of the drive sleeve 40. As shown in FIG. 16, the spring has a hook 91 at one end for attachment on the sleeve component 60. A similar hook end 92 is provided at the opposite end for attachment on the housing 10. The torsion spring 90 is pre-wound upon assembly, such that it applies a torque to the sleeve component 60 when the mechanism is at zero units dialed. The action of rotating the dose selector 80, to set a dose, rotates the sleeve component 60 relative to the housing 10, and charges the torsion spring 90 further.

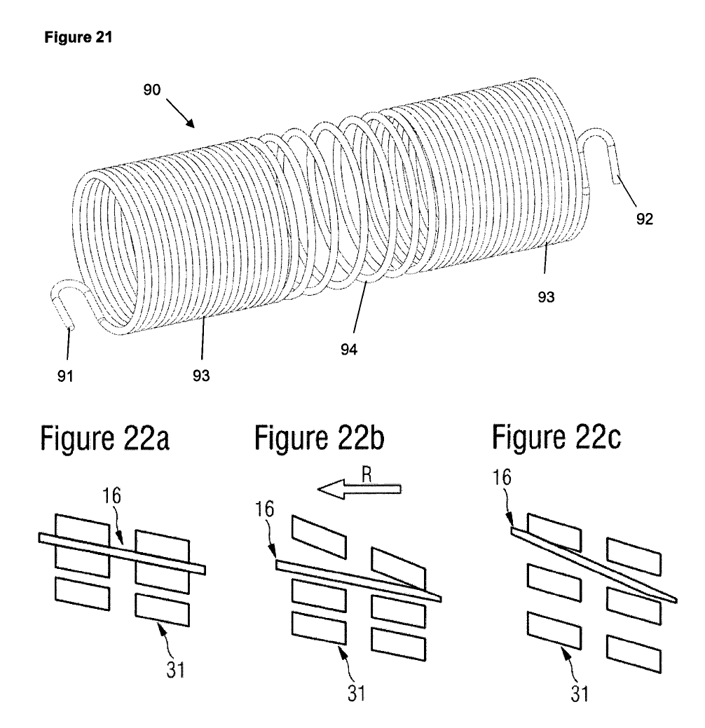

The torsion spring 90 is formed from a helical wire with at least two different pitches. In FIG. 21, both ends are formed from ‘closed’ coils 93, i.e. the pitch equals the wire diameter and each coil contacts the adjacent coil. The central portion has ‘open’ coils 94, i.e. the coils do not contact each other.

The cartridge 100 is received in cartridge holder 20 (FIG. 3). The cartridge 100 may be a glass ampoule having a moveable rubber bung 101 at its proximal end. The distal end of cartridge 100 is provided with a pierceable rubber seal which is held in place by a crimped annular metal band. In the embodiment depicted in the Figures, the cartridge 100 is a standard 1.5 ml cartridge. The device is designed to be disposable in that the cartridge 100 cannot be replaced by the user or health care professional. However, a reusable variant of the device could be provided by making the cartridge holder 20 removable and allowing backwinding of the piston rod 30 and the resetting of nut 50.

The nut member 110 is constrained to prevent rotation but allow translation relative to the housing 10 via a splined interface. The nut member 110 has a helical feature 111 on its inner surface which engages with the helical thread cut in the sleeve component 60 such that rotation of the sleeve component 60 causes axial translation of the nut member 110. This helical feature on the nut member 110 also creates a maximum dose stop abutment 112 and a zero dose abutment 113 against the end of the helical cut in the sleeve component 60 to limit the minimum and maximum dose that can be set.

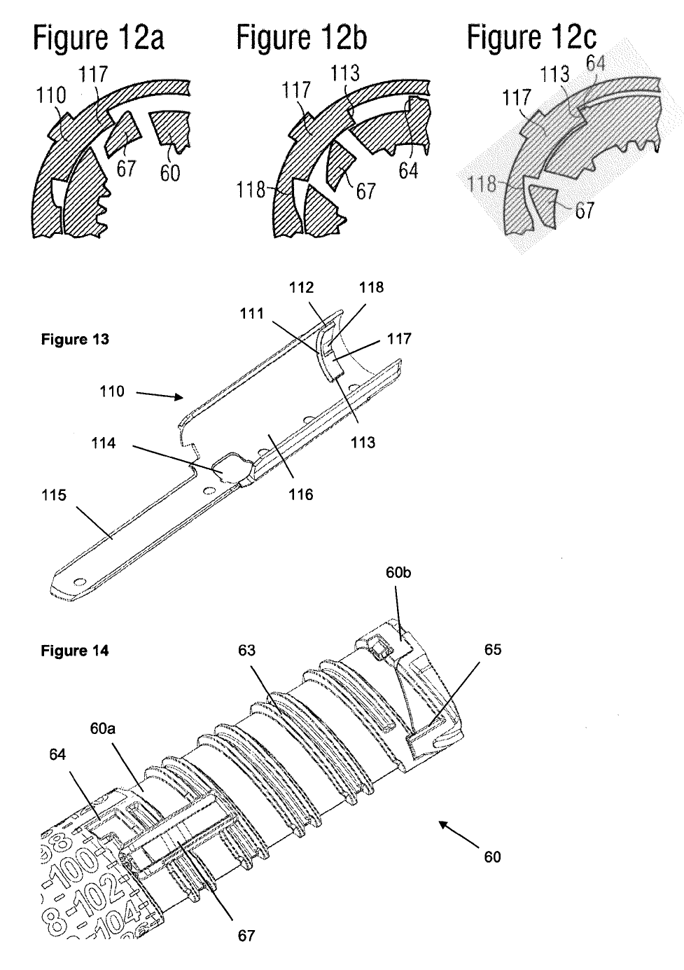

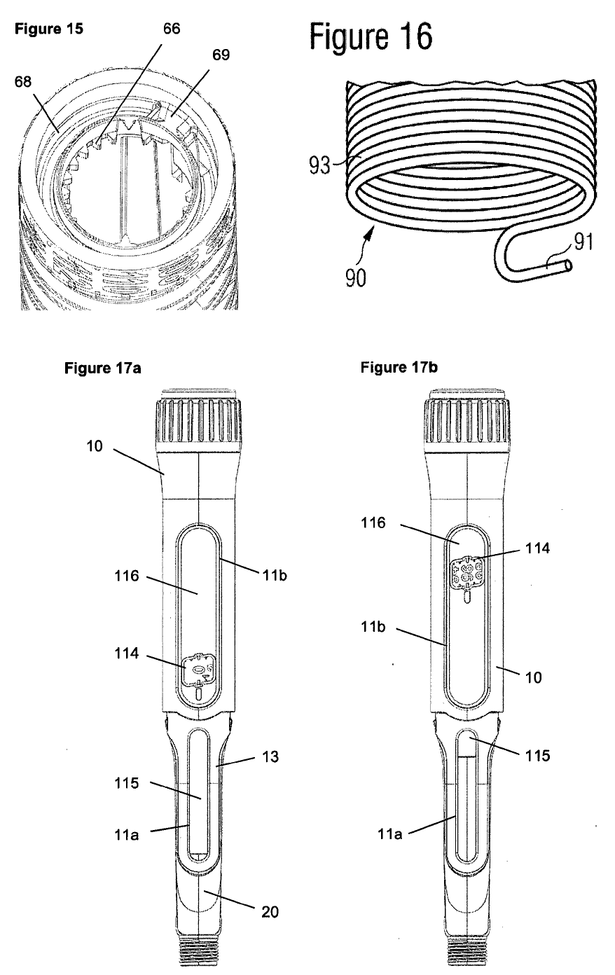

The nut member 110 has a generally plate or band like component having a central aperture 114 or window and two flanges 115, 116 extending on either side of the aperture. The flanges 115, 116 are preferably not transparent and thus shield or cover the sleeve component 60, whereas the aperture 114 or window allows viewing a portion of the sleeve component lower 60a. Further, nut member 110 has a cam 117 and a recess 118 (FIGS. 11a-12c) interacting with the clicker arm 67 of the sleeve component 60 at the end of dose dispensing.

As can be seen in FIGS. 9 and 19, the clutch plate 120 is a ring-like component. The clutch plate 120 is splined to the sleeve component 60 via splines 122. It is also coupled to the drive sleeve 40 via a ratchet interface (ratchet teeth 43, 121). The ratchet provides a detented position between the sleeve component 60 and drive sleeve 40 corresponding to each dose unit, and engages different ramped tooth angles during clockwise and anti-clockwise relative rotation. A clicker arm 123 is provided on the clutch plate 120 for interaction with ratchet features 75 of the button.

The clutch spring 130 is a compression spring. The axial position of the drive sleeve 40, clutch plate 120 and button 70 is defined by the action of the clutch spring 130, which applies a force on the drive sleeve 40 in the proximal direction. This spring force is reacted via the drive sleeve 40, clutch plate 120, and button 70, and when ‘at rest’ it is further reacted through the dose selector 80 to the housing 10. The spring force ensures that the ratchet interface (ratchet teeth 43, 121) is always engaged. In the ‘at rest’ position, it also ensures that the button splines 73 are engaged with the sleeve component splines 66, and the drive sleeve teeth 41 are engaged with teeth 14 of the housing 10.

The bearing 140 is axially constrained to the piston rod 30 and acts on the bung 101 within the liquid medicament cartridge. It is axially clipped to the piston rod 30, but free to rotate. The bearing 140 comprises a disc 141 having a stem 142 extending in the proximal direction. The stem 142 has at its proximal end a convex contact surface 143. In addition, a recessed portion 144 is provided on the stem 142. The curvature of the convex contact surface 143 and the concave contact surface 33 is chosen such that the contact diameter between the bearing 140 and piston rod 30 is small to minimize the frictional losses at this interface. The design of the clip interface between bearing 140 and piston rod 30 permits the piston rod 30 to be assembled axially, from the proximal end and through the thread engagement to the housing 10, which simplifies assembly. In addition, this design allows a simple “open and shut” mold tooling for both components.

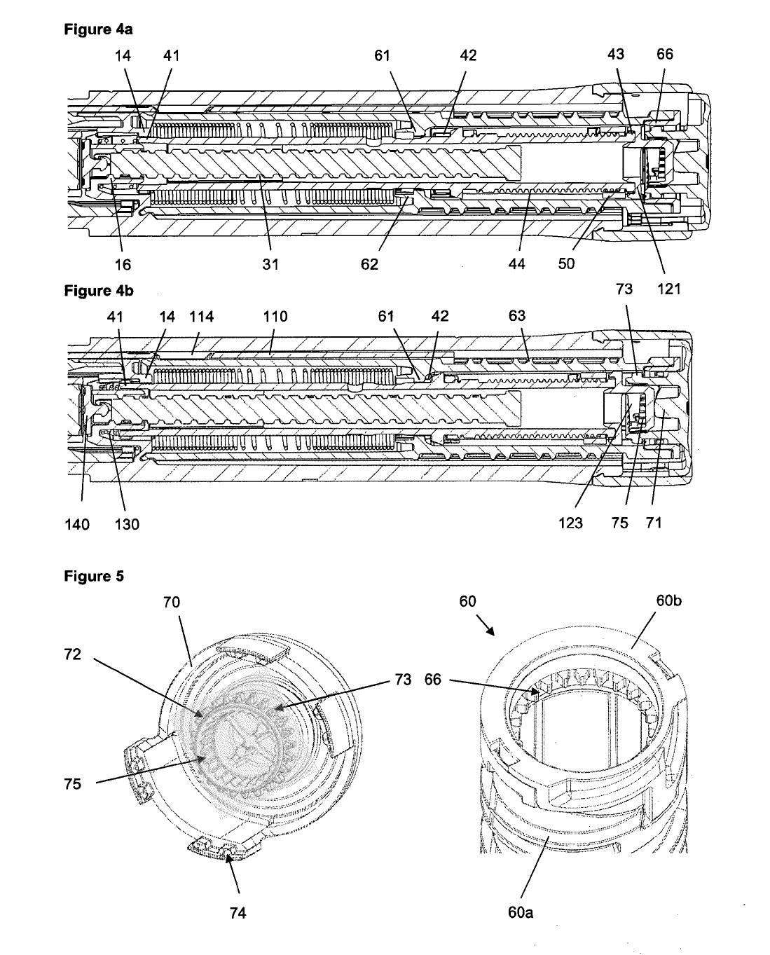

With the device in the ‘at rest’ condition as shown in FIGS. 4a and 17a, the end stop 64 of the sleeve component 60 is positioned against the zero dose abutment 113 of the nut member 110 and the button 70 is not depressed. Dose marking ‘0’ on the sleeve component 60 is visible through the windows 11b and 114 of the housing 10 and nut member 110, respectively.

The torsion spring 90, which has a number of pre-wound turns applied to it during assembly of the device, applies a torque to the sleeve component 60 and is prevented from rotating by the abutment of the end stop 64 and the zero dose abutment 113. It is also possible to ‘back-wind’ the mechanism slightly due to an offset between the abutment of the end stop 64 and the zero dose abutment 113 and the angular offset of the drive sleeve 40 spline teeth. This has the effect of preventing possible weepage when a dose is dialed and the zero dose abutment is disengaged.

The automated assembly of the torsion spring 90 into the sleeve component 60 can be achieved by incorporating large lead-ins and a groove feature to the sleeve component 60. As the torsion spring 90 is rotated during assembly, the hook end form 91 locates in the groove feature before engaging the anchor point in the sleeve component 60. To help to prevent the torsion spring 90 disengaging the anchor point 69 during subsequent assembly steps it is possible to create an interference between the torsion spring 90 and the sleeve component 60, or a one-way clip feature.

The user selects a variable dose of liquid medicament by rotating the dose selector 80 clockwise, which generates an identical rotation in the sleeve component 60. Rotation of the sleeve component 60 causes charging of the torsion spring 90, increasing the energy stored within it. As the sleeve component 60 rotates, the nut member 110 translates axially due to its threaded engagement thereby showing the value of the dialed dose. The nut member 110 has flanges 115, 116 either side of the window area 114 which cover the numbers printed on the sleeve component 60 adjacent to the dialed dose to ensure only the set dose number is made visible to the user.

A specific feature of this disclosure is the inclusion of a visual feedback feature in addition to the discrete dose number display typical on devices of this type. The distal end (flange 115) of the nut member 110 creates a sliding scale through a small window 11 a in the housing 10. As an alternative, the sliding scale could be formed using a separate component engaged with the sleeve component 60 on a different helical track.

As a dose is set by the user, the nut member 110 translates axially, the distance moved proportional to the magnitude of the dose set. This feature gives clear feedback to the user regarding the approximate size of the dose set. The dispense speed of an auto-injector mechanism may be higher than for a manual injector device, so it may not be possible to read the numerical dose display during dispense. The gauge feature provides feedback to the user during dispense regarding dispense progress without the need to read the dose number itself. For example, the gauge display may be formed by an opaque element on the nut member 110 revealing a contrasting colored component underneath. Alternatively, the revealable element may be printed with coarse dose numbers or other indices to provide more precise resolution. In addition, the gauge display simulates a syringe action during dose set and dispense.

The openings 11a, 11b in the housing 10 allow the user to view the gauge feature and number display as shown in FIGS. 17a and 17b. To reduce dust ingress and prevent the user from touching moving parts, these openings 11a, 11b are covered by translucent windows. These windows may be separate components, but in this embodiment they are incorporated into the housing 10 using ‘twin-shot’ molding technology. A first shot of translucent material forms the internal features and the windows 11a, 11b, and then a ‘second shot’ of opaque material forms the outer cover of the housing 10.

The mechanism utilizes a dose selector 80 with an increased diameter relative to the housing 10 which aids dialing although this is not a requirement of the mechanism. This feature is particularly useful (but not essential) for an auto-injector mechanism where a power supply is charged during dose setting and the torque required to turn the dose selector 80 may be higher than for a non-auto injector device.

The drive sleeve 40 is prevented from rotating as the dose is set and the sleeve component 60 rotated, due to the engagement of its splined teeth 41 with teeth 14 of the housing 10. Relative rotation must therefore occur between the clutch plate 120 and drive sleeve 40 via the ratchet interface 43, 121.

The user torque required to rotate the dose selector 80 is a sum of the torque required to wind up the torsion spring 90, and the torque required to overhaul the ratchet interface 43, 121. The clutch spring 130 is designed to provide an axial force to the ratchet interface 43, 121 and to bias the clutch plate 120 onto the drive sleeve 40. This axial load acts to maintain the ratchet teeth engagement of the clutch plate 120 and drive sleeve 40. The torque required to overhaul the ratchet 43, 121 in the dose set direction is a function of the axial load applied by the clutch spring 130, the clockwise ramp angle of the ratchet teeth 43, 121, the friction coefficient between the mating surfaces and the mean radius of the ratchet interface 43, 121.

As the user rotates the dose selector 80 sufficiently to increment the mechanism by one increment, the sleeve component 60 rotates relative to the drive sleeve 40 by one ratchet tooth. At this point the ratchet teeth 43, 121 re-engage into the next detented position. An audible click is generated by the ratchet re-engagement, and tactile feedback is given by the change in torque input required.

Relative rotation of the sleeve component 60 and the drive sleeve 40 is allowed as splines 42, 61 are disengaged during dose setting. This relative rotation also causes the last dose nut 50 to travel along its threaded path, towards its last dose abutment on the drive sleeve 40.

With no user torque applied to the dose selector 80, the sleeve component 60 is now prevented from rotating back under the torque applied by the torsion spring 90, solely by the ratchet interface 43, 121 between the clutch plate 120 and the drive sleeve 40. The torque necessary to overhaul the ratchet in the anti-clockwise direction is a function of the axial load applied by the clutch spring 130, the anti-clockwise ramp angle of the ratchet, the friction coefficient between the mating surfaces and the mean radius of the ratchet features. The torque necessary to overhaul the ratchet must be greater than the torque applied to the sleeve component 60 (and hence clutch plate 120) by the torsion spring 90. The ratchet ramp angle is therefore increased in the anti-clockwise direction to ensure this is the case whilst ensuring the dial-up torque is as low as possible.

The user may now choose to increase the selected dose by continuing to rotate the dose selector 80 in the clockwise direction. The process of overhauling the ratchet interface 43, 121 between the sleeve component 60 and drive sleeve 40 is repeated for each dose increment. Additional energy is stored within the torsion spring 90 for each dose increment and audible and tactile feedback is provided for each increment dialed by the re-engagement of the ratchet teeth. The torque required to rotate the dose selector 80 increases as the torque required to wind up the torsion spring 90 increases. The torque required to overhaul the ratchet in the anti-clockwise direction must therefore be greater than the torque applied to the sleeve component 60 by the torsion spring 90 when the maximum dose has been reached.

If the user continues to increase the selected dose until the maximum dose limit is reached, the sleeve component 60 engages with its end stop 65 on the maximum dose stop abutment 112 of nut member 110. This prevents further rotation of the sleeve component 60, clutch plate 120 and dose selector 80.

Depending on how many increments have already been delivered by the mechanism, during selection of a dose, the last dose nut 50 may contact its last dose abutment 51 with stop face 46 of the drive sleeve 40. The abutment prevents further relative rotation between the sleeve component 60 and the drive sleeve 40, and therefore limits the dose that can be selected. The position of the last dose nut 50 is determined by the total number of relative rotations between the sleeve component 60 and drive sleeve 40, which have occurred each time the user sets a dose.

With the mechanism in a state in which a dose has been selected, the user is able to deselect any number of increments from this dose. Deselecting a dose is achieved by the user rotating the dose selector 80 anti-clockwise. The torque applied to the dose selector 80 by the user is sufficient, when combined with the torque applied by the torsion spring 90, to overhaul the ratchet interface 43, 121 between the clutch plate 120 and drive sleeve 40 in the anti-clockwise direction. When the ratchet is overhauled, anti-clockwise rotation occurs in the sleeve component 60 (via the clutch plate 120), which returns the sleeve component 60 towards the zero dose position, and unwinds the torsion spring 90. The relative rotation between the sleeve component 60 and drive sleeve 40 causes the last dose nut 50 to return along its helical path, away from the last dose abutment.

With the mechanism in a state in which a dose has been selected, the user is able to activate the mechanism to commence delivery of a dose. Delivery of a dose is initiated by the user depressing the button 70 axially in the distal direction.

When the button 70 is depressed, splines between the button 70 and sleeve component 60 are disengaged, rotationally disconnecting the button 70 and dose selector 80 from the delivery mechanism, i.e. from sleeve component 60, nut member 110 and torsion spring 90. Splines 74 on the button 70 engage with splines 15 on the housing 10, preventing rotation of the button 70 (and hence the dose selector 80) during dispense. As the button 70 is stationary during dispense, it can be used in the dispense clicker mechanism as shown in FIG. 9. A stop feature in the housing 10 limits axial travel of the button 70 and reacts any axial abuse loads applied by the user, reducing the risk of damaging internal components.

The clutch plate 120 and drive sleeve 40 travel axially with the button 70. This engages the splined tooth interface 42, 61 between the drive sleeve 40 and sleeve component 60 as shown in FIGS. 7a (splines 42, 61 disengaged) and 7b (splines 42, 61 engaged), preventing relative rotation between the drive sleeve 40 and sleeve component 60 during dispense. The splined tooth interface 41, 14 between the drive sleeve 40 and the housing 10 disengages, so the drive sleeve 40 can now rotate and is driven by the torsion spring 90 via the sleeve component 60, and clutch plate 120.

Rotation of the drive sleeve 40 causes the piston rod 30 to rotate due to their splined engagement, and the piston rod 30 then advances due to its threaded engagement to the housing 10. The sleeve component 60 rotation also causes the nut member 110 to traverse axially back to its zero position whereby the abutment of the end stop 64 and the zero dose abutment 113 stops the mechanism.

The bearing 140 is axially clipped to the piston rod 30, but free to rotate. Since the bearing 140 is in direct contact with the bung 101, it does not rotate as the piston rod 30 rotates and advances during dose dispense. As described above, the contact diameter between the bearing 140 and piston rod 30 is small to minimize the frictional losses at this interface. The design of the piston rod 30 and bearing 140 eliminates delicate clip features or large contact diameters present on previous concepts. This embodiment also allows the piston rod 30 to be assembled axially, from the proximal end and through the thread engagement to the housing 10, which simplifies assembly.

Tactile feedback during dose dispense is provided via the compliant cantilever clicker arm 123 integrated into the clutch plate 120. This arm 123 interfaces radially with ratchet features 75 on the inner surface of the button 70, whereby the ratchet tooth spacing corresponds to the sleeve component 60 rotation required for a single increment dispense. During dispense, as the sleeve component 60 rotates and the button 70 is rotationally coupled to the housing 10, the ratchet features 75 engage with the clicker arm 123 to produce an audible click with each dose increment delivered.

Delivery of a dose continues via the mechanical interactions described above while the user continues to depress the button 70. If the user releases the button 70, the clutch spring 130 returns the drive sleeve 40 to its ‘at rest’ position (together with the clutch plate 120 and button 70), engaging the splines 14, 41 between the drive sleeve 40 and housing 10, preventing further rotation and stopping dose delivery.

During delivery of a dose, the drive sleeve 40 and sleeve component 60 rotate together, so that no relative motion in the last dose nut 50 occurs. The last dose nut 50 therefore travels axially relative to the drive sleeve 40 during dialing only.

Once the delivery of a dose is stopped, by the sleeve component 60 returning to the zero dose abutment, the user may release the button 70, which will re-engage the spline teeth 14, 41 between the drive sleeve 40 and housing 10. The mechanism is now returned to the ‘at rest’ condition.

It is possible to angle the spline teeth 14, 41 on either the drive sleeve 40 or housing 10 so that when the button 70 is released the re-engagement of the spline teeth 14, 41 fractionally ‘backwinds’ the drive sleeve 40 thereby removing the engagement of the sleeve component 60 to the zero dose stop abutment on the nut member 110. This compensates for the effect of clearances in the mechanism (for example due to tolerances) which could otherwise lead to slight advancement of the piston rod 30 and medicament dispense when the device is dialed for the subsequent dose due to the sleeve component 60 zero dose stop not restraining the mechanism and instead the restraint returning to the splines between the drive sleeve 40 and housing 10.

At the end of dose dispensing, additional audible feedback is provided in the form of a ‘click’, distinct from the ‘clicks’ provided during dispense, to inform the user that the device has returned to its zero position via the interaction of the clicker arm 67 on the sleeve component 60 with the ramp 47 on the drive sleeve 40 and the cam 117 and the recess 118 on the nut member 110. This embodiment allows feedback to only be created at the end of dose delivery and not created if the device is dialed back to, or away from, the zero position.

FIG. 11a shows the position of the click features when the device is in the ‘at rest’ condition, with zero units dialed and the button 70 not depressed. It can be seen that the cam feature 117 on the nut member 110 does not contact the clicker arm 67 on the sleeve component 60 when the button 70 is in the ‘at rest’ condition, so during storage or dialing the clicker arm 67 is not deflected.

During dialing, the nut member 110 translates in the proximal direction, so the cam 117 is no longer aligned axially with the clicker arm 67. At the start of dose delivery when the drive sleeve 40 translates in the distal direction, the ramp 47 on the drive sleeve 40 pushes the clicker arm 67 radially outwards. During dose delivery, the nut member 110 translates back in the distal direction, and towards the end of dose delivery, the clicker arm 67 contacts the cam 117 on the nut member 110. For small doses, the cam 117 and clicker arm 67 will be in contact at the start of the dose. FIGS. 11b to 12c show the component interactions. After dose delivery, the button 70 is released and the end of dose mechanism returns to its ‘at rest’ position.

In FIG. 11b a dose is dialed and approximately one full dial turn is applied to sleeve component 60. Nut member 110 is axially translated away from zero-unit position, so that cam 117 is no longer aligned axially with clicker arm 67. FIG. 11c shows the start of dispensing, when button 70 is depressed to initiate dose dispense and which causes the drive sleeve 70 to translate axially. Ramp 47 on the drive sleeve 40 pushes clicker arm 67 radially out and into radial alignment with cam 117 on the nut member 110.

FIG. 12a shows the mechanism at the end of dose dispensing with approximately 4 units remaining. The nut member 110 returns axially towards its zero-unit position, so that cam 117 aligns axially with clicker arm 67. Rotation of sleeve component 60 causes clicker arm 67 to contact cam 117 such that clicker arm 67 is pushed radially inwards. With approximately 2 units remaining the sleeve component 60 rotates further and clicker arm 67 follows the profile of cam 117 (FIG. 12b). This radial deflection ‘charges’ clicker arm 67 storing elastic energy. In FIG. 12c dispensing is completed as the sleeve component 60 reaches its zero-unit rotational position. The clicker arm 67 drops off the sharp edge of cam 117 into recess 118. Elastic energy is released causing clicker arm 67 to spring radially outwards to contact cam 117 and create a distinct ‘click’.

In the principal embodiment of this disclosure, the piston rod 30 advances by a fixed displacement for each revolution of the drive sleeve 40. In other embodiments, the rate of displacement may vary. For example, the piston rod 30 may advance a large displacement per revolution to dispense a first amount of medicament from the cartridge 100 and then a smaller displacement per revolution to dispense the rest of the cartridge 100. This is advantageous, as it can compensate for the fact that the first dose dispensed from the cartridge 100 often has a lower volume than other doses, for a given displacement of the mechanism.

FIG. 22 shows three embodiments with the threads 16 of the housing 10 and the threads 31 of the piston rod 30 projected around the circumference. Arrow R indicates the direction of revolution of the piston rod 30 with respect to housing 10 for all three views.

View (a) shows the principal embodiment, where the pitch is equal on the housing 10 and piston rod 30, so the piston rod 30 advances a fixed amount for every revolution of the drive sleeve 40. In view (b), the first turn of thread 31 on the piston rod 30 has a large pitch, and the other turns have a small pitch. During the first revolution, the piston rod 30 displacement depends on the large pitch of the first turn of thread 31 on the piston rod 30, so it displaces a large amount per revolution. For subsequent revolutions the piston rod 30 displacement depends on the smaller pitch of the piston rod thread 31, so it displaces a smaller amount. In view (c), the housing 10 thread 16 has a larger pitch than the piston rod 30. During the first revolution, the piston rod 30 displacement depends on the pitch of the housing thread 16, so it displaces a large amount per revolution. For subsequent revolutions the piston rod 30 displacement depends on the pitch of the piston rod thread 31, so it displaces a smaller amount.

In the previously described embodiments, a maximum settable dose is limited by a maximum potential dial extension of the nut member 110 along the sleeve component 60. In particular, the maximum settable dose is set after the sleeve component 60 has been rotated relative the housing 10 a predetermined number of times such that the sleeve component 60 engages with its end stop 65 on the maximum dose stop abutment 112 of nut member 110. This prevents further rotation of the sleeve component 60, the clutch plate 120 and the dose selector 80, thereby preventing the setting of a higher dose.

In preferred embodiments, the value of the maximum settable dose can be limited further. In particular, in the preferred embodiments, the drug delivery device is configured to allow varying the number of the maximum settable dose. This is achieved by allowing to vary the total amount of possible rotation of the sleeve component 60 relative to the housing 10.

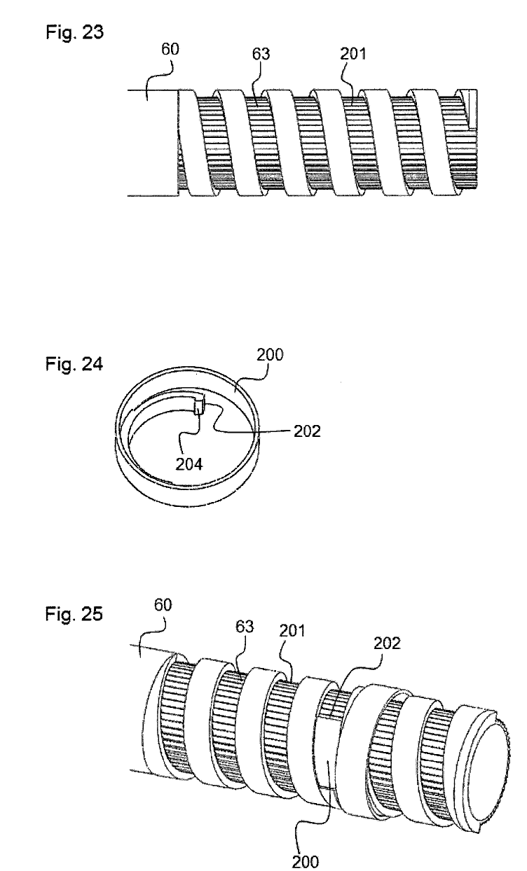

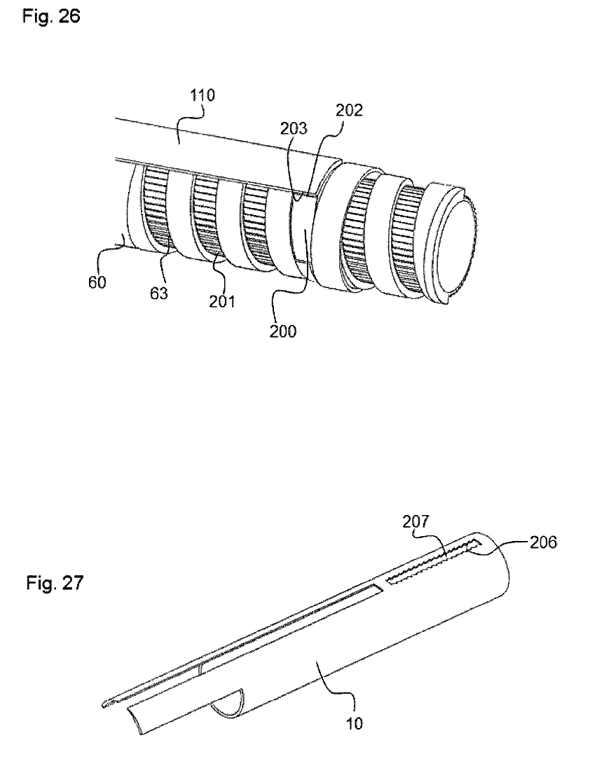

FIGS. 23 to 26 show a first preferred embodiment. In particular, FIG. 23 shows an enlarged view of a part of a sleeve component 60 according to the first preferred embodiment. Further, FIG. 24 shows a perspective view of a stop member 200 according to the first preferred embodiment. Furthermore, FIG. 25 shows the stop member 200 being assembled to the sleeve component 60. Moreover, FIG. 26 shows the nut member 110 interacting with the stop member 200 thereby limiting the maximum settable dose.

The sleeve component 60 according to the first preferred embodiment differs from the previously described sleeve component 60 in that ratchet features 201 are provided along its thread 63. In particular, the ratchet features 201 are ratchet teeth. Moreover, the end stop 65 which is molded into the sleeve component 60 in the previously described embodiments has been replaced. Instead, the stop member 200 is provided as a separate component. The stop member 200 is engageable with the sleeve component 60.

The stop member 200 is shown in FIG. 24. The stop member 200 is ring-shaped. The stop member 200 comprises an abutment surface 202 which is configured to be abutted by a corresponding abutment surface 203 of the nut member 110 as shown in FIG. 26.

Moreover, the stop member 200 comprises a ratchet feature 204 which is configured for engaging the ratchet features 203 on the thread 63 of the sleeve component 60. The ratchet feature 204 of the stop member 200 is a ratchet arm. The ratchet feature 204 of the stop member 200 and the ratchet feature 201 of the sleeve component 60 are configured to allow a rotation of the stop member 200 along the thread 63 in a first rotational direction and to prevent a rotation of the stop member 200 along the thread 63 in a second rotational direction opposite to the first rotational direction when engaged with each other. The first rotational direction is a clockwise direction and the second rotational direction is an anti-clockwise direction. In an alternative embodiment, the first rotational direction is an anti-clockwise direction and the second rotational direction is a clockwise direction.

FIG. 25 shows the stop member 200 being assembled to the sleeve component 60. The stop member 200 is threaded clockwise onto the sleeve component 60 from the proximal end of the sleeve component 60 up to a desired position which defines the maximum settable dose. In particular, the stop member 200 is threaded onto the sleeve component 60 during the assembly of the drug delivery device. After the assembly has been completed, the position of the stop member 200 relative to the sleeve component 60 cannot be altered anymore. For example, the stop member 200 may be covered by the housing 10 such that the stop member 200 is not accessible to a user of the drug delivery device.

FIG. 26 shows the assembly comprising the sleeve component 60, the stop member 200 and the nut member 110 during a dose setting operation wherein the maximum settable dose is set. For the sake of clarity, other components of the drug delivery device are not shown in FIG. 26.

During the dose setting operation, the sleeve component 60 is rotated relative to the nut member 110 such that the nut member 110 travels along the thread 63 of the sleeve component 60, thereby moving in the proximal direction relative to the sleeve component 60. The nut member 110 is configured to be advanced proximally up until the maximum dose stop abutment 112 of the nut member 110 abuts with the stop member 200, as shown in FIG. 26. In particular, the maximum dose stop abutment 112 of the nut member 110 abuts with the abutment surface 203 of the stop member 200.

The engaged ratchet features 201, 204 of the stop member 200 and of the sleeve component 60 prevent the stop member 200 from threading off the sleeve component 60. Thus, the stop member 200 is prevented from being moved further in the proximal direction by the nut member 110 due to the engagement of the ratchet features 201, 204 of the stop member 200 and of the sleeve component 60.

Accordingly, the stop member 200 acts as a stop, preventing a further movement of the nut member 110 along the thread 63, and thereby preventing setting of a higher dose.

In particular, the axial position of the stop member 200 relative to the sleeve component 60 determines the possible range of relative movements of the sleeve component 60 and the nut member 110. Thereby, the stop member 200 limits a rotation of the sleeve component 60 relative to the nut member 110 and also relative to the housing 10 to a total amount of possible rotation. The maximum settable dose depends on said total amount of possible rotation.

In the first preferred embodiment shown in FIGS. 23 to 26, the position of the stop member 200 relative to the sleeve component 60 is set during assembly of the drug delivery device. This is done by the manufacturer of the drug delivery device.

This concept allows the use of the same components on the same assembly line to produce drug delivery devices differing in their respective maximum settable dose. In particular, the maximum settable dose can be set to a large variety of values depending on the position of the stop member 200 on the thread 63. Therefore, the drug delivery device can be used for multiple medicaments or drug regimens with minimal implications or alterations required to the design of the parts, the logistics or the assembly line. In particular, the manufacture can vary the position of the stop member 200 along the thread 63, thereby varying the total amount of possible rotation of the sleeve component 60 relative to the housing 10.

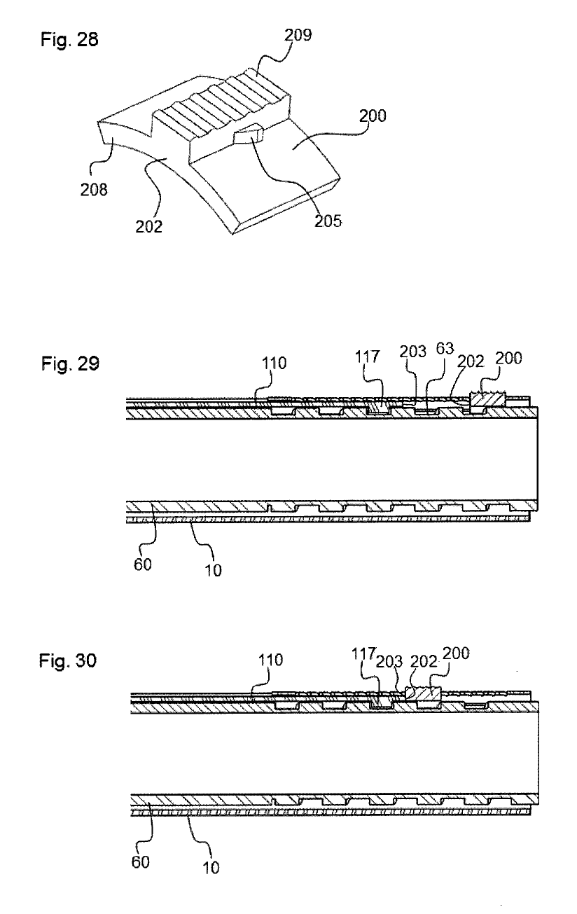

FIGS. 27 to 30 show a second preferred embodiment which also allows varying the maximum settable dose by varying the total amount of possible rotation of the sleeve component 60 relative to the housing 10 and relative to the nut member 110. In particular, FIG. 27 shows a perspective view of the housing 10 according to the second preferred embodiment. FIG. 28 shows the stop member 200 according to the second preferred embodiment. FIG. 29 and FIG. 30 each show a cross-sectional view of the assembly comprising the sleeve component 60, the stop member 200 and the nut member 110. For the sake of clarity, other components of the drug delivery device are not shown in FIGS. 29 and 30.

The stop member 200 according to the second preferred embodiment is configured to directly engage with the housing 10. The stop member 200 comprises a first engagement feature 205. The first engagement 205 feature is a tooth. In particular, the first engagement 205 comprises two teeth provided on the stop member 200.

Further, the housing 10 comprises a slot 207 extending axially. Moreover, the housing 10 comprises a second engagement feature 206. The second engagement feature 206 is a set of ratchet features which are arranged in the slot 207 of the housing 10. The stop member 200 can be engaged with the slot 207 of the housing 10. In particular, the first engagement feature 205 of the stop member 200 is engageable with the second engagement feature 206 of the housing 10.

When the stop member 200 is engaged with the slot 207, an inner part 208 of the stop member 200 protrudes into the inside of the housing 10.

The abutment surface 202 of the stop member 200 is a distally facing surface. In particular, the abutment surface 200 is arranged on the inner part 208 of the stop member 200 which protrudes into the inside of the housing 10 when the stop member 200 is engaged with the housing 10.

When the stop member 200 is engaged with the housing 10, the position of the stop member 200 relative to the housing 10 defines the maximum settable dose by limiting the total amount of possible rotation of the sleeve component 60 relative to the housing 10, as will be discussed in further detail with respect to FIGS. 29 and 30.

Moreover, the stop member 200 comprises an outer part 209 which protrudes out of the housing 10 when the stop member 200 is engaged with the housing 10.

The first and the second engagement features 205, 206 are configured to allow an axial movement of the stop member 200 relative to the housing 10 when the first and the second engagement features 205, 206 are engaged. However, only an axial movement in the distal direction is allowed. The first and the second engagement feature 205, 206 prevent an axial movement of the stop member 200 relative to the housing 10 in the proximal direction. For this purpose, the first and the second engagement features 205, 206 are ratchet features having tapered surfaces. Accordingly, once the stop member 200 is engaged with the housing 10, it can only be moved in the distal direction relative to the housing 10, not in the proximal direction.

The stop member 200 is accessible from an outside of the drug delivery device. For example, a health care professional can move the stop member 200 relative to the housing 10, thereby determining the maximum settable dose.

FIGS. 29 and 30 show the process of setting a maximum settable dose. The maximum settable dose could be set by initially dialing the device up to where the maximum settable dose should be, as shown in the cross-sectional view of FIG. 29. This is followed by advancing the stop member 200 from an initial position at the proximal end of the slot 207 provided in the housing 10 up until the stop member 200 abuts the nut member 100, as shown in FIG. 30. In particular, the distally facing abutment surface 202 of the stop member 200 may abut the corresponding abutment surface 203 of the nut member 110.

As the stop member 200 is prevented from moving proximally relative to the housing 10 due to the engagement of the first and the second engagement feature 205, 206, the movement of the nut member 110 relative to the housing 10 is also limited. In particular, the nut member 110 cannot be moved any further axially in the proximal direction relative to the housing 10. As the nut member 110 can only carry out a concurrently axial and rotational movement relative to the housing 10, a further rotational movement of the nut member 110 in one rotational direction relative to the housing 10 is also prevented. Thereby, increasing the set dose beyond this value is also prevented.

Prior to delivering the drug delivery device to a user, the nut member 110 could be unwound back to its initial, most distal position.

As the stop member 200 is configured to be moved from its initial position at the distal end of the slot 207 in the housing 10 into other positions along the slot 207, the distance by which the nut member 110 is enabled to move relative to the sleeve component 60 can be varied. Thereby, the total amount of possible rotation of the sleeve component 60 relative to the housing 10 is varied. In particular, the total amount of possible rotation of the sleeve component 60 relative to the housing 10 can be varied after the assembly of the drug delivery device has been completed.

Additionally, means to tamper-proof the setting of the stop member 200 could be provided. Said means to tamper-proof could prevent a user from changing the position of the stop member 200.

In an alternative design, the stop member 200 does not comprise an outer part 209 which protrudes from the slot 207 in the housing 10. Instead, the stop member 200 may be an internal member which is not directly accessible from the outside of the drug delivery device. Further, the health care professional could be provided with a specific tool to manipulate the position of the stop member 200. In particular, without the specific tool, it may not be possible to adjust the position of the stop member 200. Furthermore, the stop member 200 and the slot 207 in the housing 10 could be concealed, i.e. by the application of a label or similar, to further prevent the user from manipulating the stop member 200.

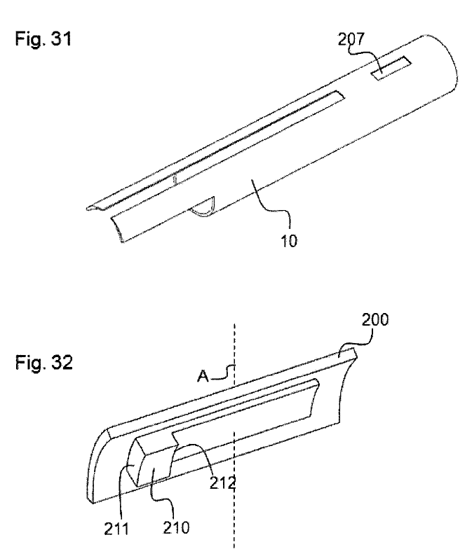

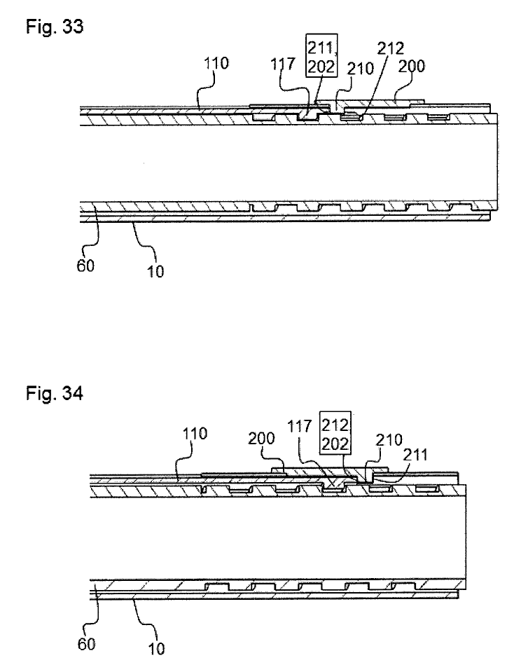

FIGS. 31 to 34 show a third preferred embodiment. In particular, FIG. 31 shows a perspective view of the housing 10 according to the third preferred embodiment. FIG. 32 shows the stop member 200 according to the third preferred embodiment. Further, FIGS. 33 and 34 each show cross-sectional views of the assembly comprising the housing 10, the nut member 110, the sleeve component 60 and a stop member 200. Again, other components of the drug delivery device are not shown in order to increase the clarity of FIGS. 33 and 34.

According to the third preferred embodiment, the housing 10 also comprises a slot 207 extending in the axial direction. The slot 207 is free from ratchet features. Instead, the slot 207 comprises engagement features (not shown) allowing a clipping of the stop member 200 to the housing 10. The stop member 200 is configured to be engaged with the housing 10 by clipping. In particular, the stop member 200 is configured to be clipped into the slot 207 provided in the housing.

The stop member 200 comprises a protrusion 210 which is arranged at an inner surface of the stop member and which protrudes inwardly when the stop member 200 is engaged with the housing 10.

The maximum settable dose is determined by the position of the protrusion 210. When the stop member 200 is engaged with the housing 10, the protrusion 210 protrudes through the housing 10 up to the outer surface of the sleeve component 60. The nut member 110 is configured to travel along the thread 63 of the sleeve component 60. Thereby, the protrusion 210 of the stop member 200 establishing an end stop for the nut member 110, preventing the nut member 110 from advancing further proximally during a dose setting operation.

The stop member 200 is configured such that it can be engaged with the housing 10 in two different axial orientations. The stop member shown in FIG. 32 is configured to be engaged with the housing 10 either in a first orientation or in a second orientation wherein the stop member 200 is moved from its first orientation to its second orientation by being rotated by 180° around a rotation axis A which is indicated in FIG. 32 by the dotted line.

For example, the stop member 200 shown in FIG. 32 is configured to set a maximum dose stop either at one third or at two thirds of the maximum potential dial extension. The protrusion 210 comprises two axially facing surfaces 211, 212. When the stop member 200 is engaged with the housing 10 in the first orientation, the surface 211 becomes the abutment surface 202 of the stop member 200 configured to be abutted by the nut member 110. If the stop member 200 is rotated around the rotational axis A by 180°, the surface 212 becomes the abutment surface 202 of the stop member 200 configured to be abutted by the nut member 110. In general, the one of the surfaces 211, 212 which faces distally is the abutment surface 202 of the stop member 200 configured to be abutted by the nut member 110.

FIG. 33 shows the stop member in a state wherein it is engaged with the housing such that the maximum settable dose is set to one third of the maximum potential dial extension, i.e. in the first orientation. FIG. 34 shows the stop member 200 engaged with the housing 10 in the second orientation, thereby setting the maximum settable dose to two thirds of the maximum potential dial extension.

Accordingly, the stop member 200 allows varying the total amount of possible rotation of the sleeve component 60 relative to the housing 10 by varying the orientation in which the stop member 200 is engaged with the housing 10. A variation of the orientation results in a movement of the abutment surface 202, 211, 212 relative to the sleeve component 60, thus increasing or decreasing the distance by which the nut member 110 is allowed to move relative to the sleeve component 60.

Moreover, according to the third preferred embodiment, at least a second stop member may be provided with the drug delivery device wherein the second stop member differs from the stop member 200 in the position of the distally facing abutment surface. Accordingly, the second stop member may define a different distance by which the nut member 110 is allowed to move relative to the sleeve component 60.

In particular, the drug delivery device could be supplied with multiple stop members 200 so that the maximum dose setting can be set to a variety of values. A health care professional may set the maximum settable dose by engaging the corresponding stop member 200 to the housing 10. In particular, depending on the desired maximum settable dose, a corresponding stop member 200 can be chosen and engaged with the housing. The stop members 200 may differ in the axial position of their respective abutment surface 202.

In an alternative design of this concept, the axial movement of the nut member 110 is blocked by an abutment of surfaces at a position different from the proximal end face of the nut member 110. For example, the axially facing abutment surface 202 of the stop member 200 may be arranged to engage with a corresponding surface in close proximity to a distal end of the nut member 110.

For example, a lens covering the opening 11a or 11b in the housing 10 may be designed as the stop member 200. The lens may comprise an inwardly protruding surface configured to engage with a corresponding surface of the nut member 110. The lens may be engageable with the housing 10 at least in two different orientations. The lens could be inserted during the assembly of the device so that the total number of parts is not increased. Alternatively, a health care professional could be provided with a selection of lenses, each lens defining a different total amount of possible rotation. The appropriate lens for a given medicament or a user's maximum dose requirement could be clipped into the housing from the outside, e.g. by the health care professional.

### REFERENCE NUMERALS

10 housing

11a, b opening

12 flange-like inner wall

13 strip

14 teeth

15 spline

16 inner thread

20 cartridge holder

30 piston rod

31 outer thread

32 clip arm

33 concave contact surface

40 drive sleeve

41 teeth

42 spline

43 ratchet teeth

44 threaded section

45 spline

46 last dose stop

47 ramp

50 nut

51 last dose stop

52 spline

60 sleeve component

60a sleeve component lower

60b sleeve component upper

61 spline

62 flange

63 outer thread

64, 65 end stop

66 spline

67 clicker arm

68 groove

69 anchor point

70 button

71 stem

72 flange

73, 74 spline

75 ratchet teeth

80 dose selector

90 torsion spring

91, 92 hook

93, 94 coil

100 cartridge

101 bung

110 nut member

111 helical feature

112 maximum dose stop abutment

113 zero dose abutment

114 aperture

115, 116 flange

117 cam

118 recess

120 clutch plate

121 ratchet teeth

122 protrusion

123 clicker arm

130 clutch spring

140 bearing

141 disc

142 stem

143 convex contact surface

144 recessed portion

200 stop member

201 ratchet feature of the number sleeve

202 abutment surface of the stop member

203 corresponding abutment surface of the nut member

204 ratchet feature of the stop member

205 first engagement feature of the stop member

206 second engagement feature of the housing

207 slot

208 inner part of the stop member

209 outer part of the stop member

210 protrusion of the stop member

211 axially facing surface

212 axially facing surface

A rotation axis

I longitudinal axis

R direction of revolution

1. A mechanism for setting a dose of a medicament for a variable dose drug delivery device, comprising:
a housing;
a sleeve component configured to rotate relative to the housing during a dose setting operation; and
a stop member configured to limit a total amount of possible rotation of the sleeve component to a maximum in order to limit a maximum settable dose,
wherein the stop member is configured to allow varying the total amount of possible rotation of the sleeve component.

2. The mechanism according to claim 1,
wherein the sleeve component comprises a thread,
wherein the mechanism comprises a nut member that is engaged with the thread such that the nut member is configured to travel along the thread during the dose setting operation, and
wherein the stop member is configured to limit a rotation of the sleeve component by limiting a movement of the nut member relative to the sleeve component.

3. The mechanism according to claim 2, wherein the stop member is configured to directly engage with the nut member.

4. The mechanism according to claim 2,
wherein the stop member is engaged with the thread of the sleeve component,
wherein the total amount of possible rotation of the sleeve component relative to the housing is determined by the position of the stop member along the thread, and
wherein the total amount of possible rotation can be varied by moving the stop member along the thread.

5. The mechanism according to claim 2, wherein the stop member and the sleeve component each comprise a ratchet feature allowing a rotation of the stop member along the thread during manufacture of the mechanism and preventing a rotation of the stop member in an assembled drug delivery device.

6. The mechanism according to claim 2, wherein the stop member and the sleeve component each comprise a ratchet feature allowing a rotation of the stop member along the thread in a first rotational direction and preventing a rotation of the stop member in a second rotational direction opposite to the first rotational direction.

7. The mechanism according to claim 2,
wherein the stop member comprises an abutment surface, and
wherein the stop member is configured to limit an axial movement of the nut member in a first axial direction relative to the sleeve component when the nut member abuts the abutment surface of the stop member.

8. The mechanism according to claim 7,
wherein the abutment surface of the stop member is arranged such that the abutment surface is configured to be abutted by an end face of the nut member that faces in a circumferential direction, or
wherein the abutment surface of the stop member is arranged such that the abutment surface is configured to be abutted by a protrusion of the nut member protruding in a radially outward direction.

9. The mechanism according to claim 1, wherein the stop member is configured to be engaged with the housing in two different orientations, wherein each orientation corresponds to a different total amount of possible rotation.

10. The mechanism according to claim 1,
wherein the stop member comprises a first engagement feature and the housing comprises a second engagement feature,
wherein the first engagement feature of the stop member is directly engagable with the second engagement feature of the housing, and
wherein the first and the second engagement features are configured to allow an axial movement of the stop member relative to the housing when the first and the second engagement features are engaged.

11. The mechanism according to claim 10, wherein the first and the second engagement features are configured to allow an axial movement of the stop member in a second axial direction relative to the housing and to prevent an axial movement of the stop member in a first axial direction relative to the housing.

12. The mechanism according to claim 7, further comprising at least a second stop member comprising a second abutment surface,
wherein the housing is configured to be engaged with exactly one of the stop members at a given time, and
wherein each of the stop members defines a different total amount of possible rotation of the sleeve component relative to the housing depending on an axial position of the abutment surface of the respective stop member.

13. The mechanism according to claim 1, wherein the stop member is configured such that the total amount of possible rotation can only be varied by engaging the stop member with a specific tool.

14. The mechanism according to claim 1,
wherein the stop member is retained inside the housing, or
wherein the stop member is configured to be assembled to the remainder of the mechanism from the outside of the housing.

15. A drug delivery device comprising a mechanism for setting a dose of a medicament for the drug delivery device, the mechanism comprising:
a housing;
a sleeve component configured to rotate relative to the housing during a dose setting operation; and
a stop member configured to limit a total amount of possible rotation of the sleeve component to a maximum in order to limit a maximum settable dose,
wherein the stop member is configured to allow varying the total amount of possible rotation of the sleeve component.
